(19) United States
(12) Patent Application Publication
(10) Pub. No.: US 2019/0201207 A1
(43) Pub. Date: Jul. 4, 2019

(54) PROXIMAL CARPAL ROW IMPLANT

(71) Applicant: Guilherme Giusti, Eau Claire, WI (US)

(72) Inventor: Guilherme Giusti, Eau Claire, WI (US)

(21) Appl. No.: 16/334,186

(22) PCT Filed: Sep. 18, 2017
(86) PCT No.: PCT/US2017/052074

§ 371 Date: Mar. 18, 2019

Related U.S. Application Data
(60) Provisional application No. 62/396,442, filed on Sep. 19, 2016.

(51) Int. Cl. A61F 2/42; A61F 2/08

(52) U.S. Cl. CPC A61F 2/4261; A61F 2002/30301; A61F 2/0811

(57) ABSTRACT

Provided is single, monolithic wrist implant for the proximal carpal row.

### CROSS-REFERENCE TO RELATED APPLICATIONS

This application claims the benefit of U.S. Provisional Patent Application Ser. No. 62/396,442 filed Sep. 19, 2016, the disclosure of which is incorporated herein by reference.

### BACKGROUND

Various surgical reconstruction or replacement for the proximal row of a human carpus, wrist joint are known but there is a continuing need to provide improved wrist implants.

### SUMMARY

Disclosed are methods and systems for use as a replacement of the proximal carpal row implant. In one aspect is disclosed a wrist implant for replacement of the proximal carpal row, the implant comprising:
a single, one-piece integral body, the body having a substantially saddle-shaped or concave arcuate configuration comprising:
   a dorsal surface;
   a palmar surface;
   a proximal end having a substantially convex shape;
   a distal end configured to have three articulation surfaces and substantially opposing the proximal end, the distal end comprising;
      a first lateral articulating surface defined by a first convex portion;
      a second lateral articulating surface defined by second convex portion wherein the first lateral articulating surface is substantially opposing the second lateral articulating surface; and
      a central articulating surface defined by a concave portion.

In another aspect is disclosed a wrist implant configured to replicate a proximal carpal row of a human wrist, the implant comprising:
a single, one-piece integral body, the body having a substantially saddle-shaped or concave arcuate configuration comprising:
   a dorsal surface;
   a palmar surface;
   a proximal end having a substantially convex shape;
   a distal end configured to have three articulation surfaces and substantially opposing the proximal end, the distal end comprising;
      a first lateral articulating surface defined by a first convex portion;
      a second lateral articulating surface defined by second convex portion wherein the first lateral articulating surface is substantially opposing the second lateral articulating surface; and
      a central articulating surface defined by a concave portion.

In yet another aspect is disclosed a method for replacing a proximal carpal row of a human hand comprising:
implanting a wrist implant, the wrist implant comprising:
   a single, one-piece integral body, the body having a substantially saddle-shaped or concave arcuate configuration comprising:
   a dorsal surface;
   a palmar surface;
   a proximal end having a substantially convex shape;
   a distal end configured to have three articulation surfaces and substantially opposing the proximal end, the distal end comprising;
   a first lateral articulating surface defined by a first convex portion;
   a second lateral articulating surface defined by second convex portion wherein the first lateral articulating surface is substantially opposing the second lateral articulating surface; and
   a central articulating surface defined by a concave portion.

In still another aspect is disclosed a kit for replacing a proximal carpal row of a human hand comprising:
various sized wrist implants and for left and right hand configurations, the
wrist implant comprising:
   a single, one-piece integral body, the body having a substantially saddle-shaped or concave arcuate configuration comprising:
   a dorsal surface;
   a palmar surface;
   a proximal end having a substantially convex shape;
   a distal end configured to have three articulation surfaces and substantially opposing the proximal end, the distal end comprising;
   a first lateral articulating surface defined by a first convex portion;
   a second lateral articulating surface defined by second convex portion wherein the first lateral articulating surface is substantially opposing the second lateral articulating surface; and
   a central articulating surface defined by a concave portion.

### BRIEF DESCRIPTION OF THE DRAWINGS

FIG. 1 is a palmar view of the bones of a human hand and wrist.

FIG. 2 is plan, dorsal view of an exemplary wrist implant according to one embodiment.

FIG. 3 is coronal-sectional, dorsal view of the wrist implant of FIG. 2 implanted into a wrist according to one embodiment.

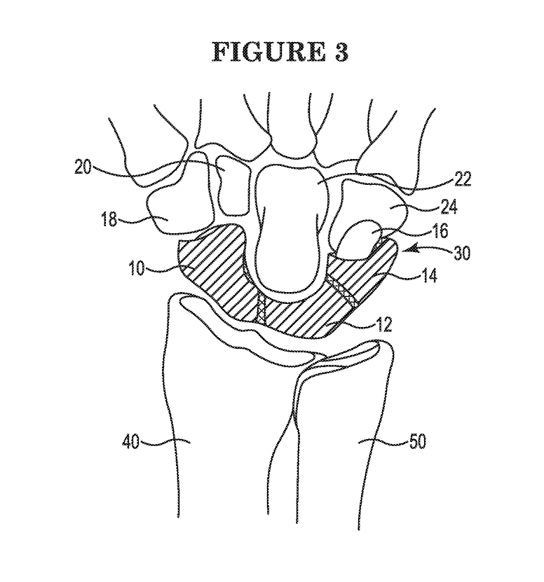

FIG. 4 is coronal-sectional, dorsal view of the wrist implant of FIG. 2 implanted into a wrist according to one embodiment.

FIG. 5 is a sagittal view of the wrist implant of FIG. 2 implanted into a wrist according to one embodiment.

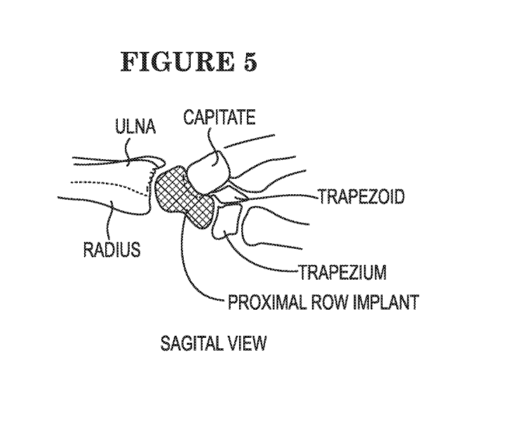

### DETAILED DESCRIPTION

Surgical treatments for arthritis and similar conditions of the wrist joint have included intercarpal fusion, wrist fusion, local resection, proximal row carpectomy, and soft tissue interposition arthroplasty. Fusion procedures affect stability, power and mobility of the wrist. Local resection procedures, which involve removal of bone, are complicated by migration of adjacent carpal bones into the space left by the resection which results in unwanted instability.

Various forms of flexible implants formed from silicone rubber have been developed to replace the lunate or scaphoid bones of the carpal row. Such implants are designed to act as articulating spacers, however, they are not capable of maintaining the relationship of adjacent carpal bones. Examples of such lunate or scaphoid implants can be found in U.S. Pat. Nos. 4,164,793; 4,198,712; 4,936,860; 4,955,915 and 4,969,908. These implants are not typically favored by most surgeons and the current surgical preference is for partial or complete bone fusion or total wrist arthroplasty.

The human hand consists of several small bones called phalanges (finger digits), metacarpals (the palm) and carpals (wrist). The forearm consists of two bones, namely, the radius and the ulna. The wrist is broadly defined as the multiple articulations of the eight carpal bones (carpus) with the neighboring hand and forearm. This complex system of articulations works in unison to provide a global range of motion for the wrist joint. Motion at the wrist joint occurs between the radius and the first (or proximal) row of carpal bones, which move essentially as a single functional unit, and between the proximal carpal row and the distal carpal row of carpal bones. There is minimal motion between the bones of the distal carpal row and the metacarpal bones of the hand.

As used herein, the directional terms reference a human hand. The term “palmar” refers to the palm of the hand. “dorsal” refers to the back of the hand. The term “proximal” refers to a portion closest to the forearm, namely the radius. The term “distal” refers to the portion furthest away from the forearm and closer to the metacarpals.

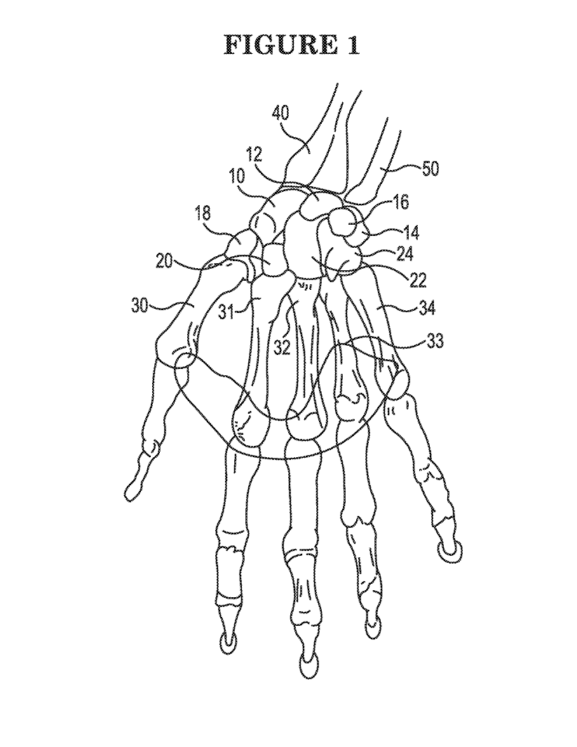

As illustrated in FIG. 1, there are eight carpal bones, namely, the scaphoid 10, lunate 12, triquetrum 14, pisiform 16, trapezium 18, trapezoid 20, capitate 22 and the hamate 24. Each carpal bone possesses a unique, highly complex three-dimensional shape. The arrangement of the eight carpal bones can be grossly described as having two rows to form a compact, powerful unit. The proximal carpal row contains the scaphoid 10 (also called the navicular), lunate 12, and triquetrum 14. These three bones articulate proximally with the radius 40 and the triangular fibrocartilage. The ulna 50 does not articulate directly with the carpus but is separated from the triquetrum 14 by the triangular fibrocartilage, which acts as a stabilizing structure.

The distal carpal row contains the trapezium 18, trapezoid 20, capitate 22, and hamate 24 and articulates distally with the five metacarpals (metacarpals bones 30, 31, 32, 33 and 34), and proximally with the three bones of the proximal carpal row. The pisiform 16 is a sesamoid bone which articulates with the triquetrum 14 alone, and does not participate directly in carpal or global wrist motion. The scaphoid 10 acts as a connecting link between the proximal and distal carpal rows and is a critical coordinator of carpal motion.

The wrist is generally divided into five primary articulations in addition to the intercarpal joint spaces: the radiocarpal joint, the midcarpal joint (between the proximal and distal carpal rows), the large carpometacarpal joint (between the distal carpal row and the second, third, fourth and fifth metacarpals (bones 31, 32, 33 and 34), the thumb carpometacarpal joint (between the first metacarpal 30 and the trapezium 18) and the distal radio-ulnar joint (DRUJ). The midcarpal joint is the joint between the scaphoid, lunate, and triquetrum proximally, and the second row of carpal bones distally and is made up of three distinct portions: in the center the head of the capitate and the superior surface of the hamate articulate with the deep cup-shaped cavity formed by the navicular and lunate, and constitute a sort of ball-and-socket joint. On the radial side (i.e. near the radius 40) of the midcarpal joint, the trapezoid 20 and trapezium 18 form a concave articulating surface with the distal scaphoid. On the ulnar side (i.e. near the ulnar 50), the convex hamate surface articulates with a helicoidal surface of the distal triquetrum. The midcarpal joint can thus be characterized as containing a number of convex and concave surfaces that interact with one another to provide the desired joint movements.

Radiocarpal ligaments limit motion between the radius and the carpal bones and intercarpal ligaments limit motion between neighboring carpal bones. The distal radioulnar joint is an articulation between the radius and the ulnar head, and is contained within a capsule-like structure of cartilage, synovial membrane and ligaments. A triangular fibrocartilage between the ulna 50 and the carpus separates the distal radioulnar joint from the rest of the wrist.

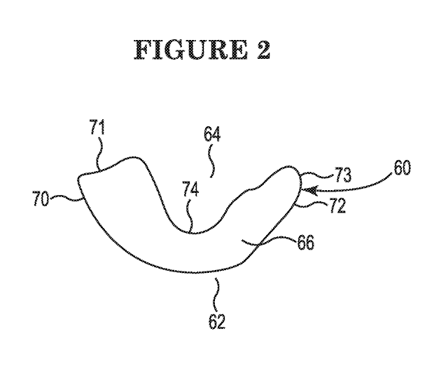

Disclosed is a wrist implant 60 for replacement of the entire proximal carpal row. As seen in FIGS. 2 & 3 and as explained in detail below, the wrist implant 60 includes the proximal carpal row as a one-piece, monolithically formed body that defines the shape of the three bones of the proximal carpal row together in their anatomic sequence and position scaphoid, lunate and triquetrum from a dorsal view. The wrist implant 60 is configured to define a proximal 62, distal 64, dorsal 66, palmar 68 (not shown), and lateral surfaces 70, 72. The dorsal and palmar surface can be substantially planar. The shape of the wrist implant 60 is substantially saddle-shaped or a concave arcuate configuration. The proximal end 62 is configured to have a convex shape. The distal end 64 configured to have three articulation surfaces and substantially opposing the proximal end 62, the distal end 64 defining a first lateral articulating surface 70 defined by a first convex portion 71 and a second lateral articulating surface 72 defined by second convex portion 73, and a central articulating surface 74 defined by a concave portion.

The first lateral articulating surface 70 defined by a first convex portion 71, which corresponds to the scaphoid side and configured to articulate with the concavity formed by the trapezium, trapezoid and/or lateral aspect of the capitate. The first lateral articulating surface can be configured to articulate with a trapezium and/or trapezoid. The central articulating surface 74 defined by a concave portion is operable to articulate with the capitate 22 and the more medial articulation is helicoid and articulates with the hamate bone 24. The second lateral articulating surface 72 defined by a second convex portion 73, which corresponds to triquetrum side and configured to articulate with the concavity formed by the hamate bone. The central articulating surface 74 defined by a concave portion is operable to articulate with the capitate 22 and the more medial articulation is helicoid and articulates with the hamate bone 24. The proximal surface 62 is operable to articulate about the radius 40.

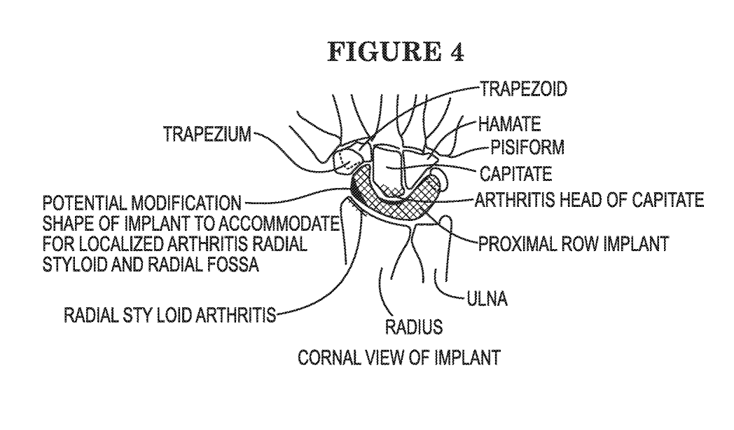

The proximal carpal row implant 60 can be customized to patient's anatomy using contralateral wrist CT scan and printing a model in a mirror image of the involved wrist. The model can be used to produce the final implant with the appropriate material. In some embodiments, the implant is available in left and right hand configurations with geometrically scaled sizes that approximate the anthropomorphic sizes of different radio-carpal joints. In some embodiments, the implant 60 can be configured to accommodate variation in a patient's surrounding anatomies. For example, if a patient's radial styloid and capitate head are affected by arthritis, the implant can include depressions or more defined recesses (shown as xxxs in FIGS. 4 & 5) to reduce load to the affected areas.

The implant 60 can be configured to define a dorsal surface which may or may not contain suture passages for ligament attachment. In some embodiments, the implant may not require any fixation to, for example, the radius or distal carpal row. The proximal carpal row works as an intercalated segment between radius and the distal carpal row keeping the proximal carpal row in position.

A dorsal ulnar passage can allow the attachment of the dorsal carpal ligaments that inserts to the dorsal aspect of the triquetrum such as the dorsal intercarpal and the radiotriquetrum ligaments; a palmar passage over the radial palmar aspect of the scaphoid can allow the attachment of palmar radial ligaments such as the radioscaphoid and radioscaphocapitate ligaments. The implant can also have a palmar surface which articulates with the pisiform bone.

The implant can be stabilized by suturing to two ligaments of the wrist, if needed. A suture is stitched to the palmar/radial ligament and the ends thereof are passed through the suture passages on the lateral/radial side. Another suture is stitched to the other dorsal/ulnar ligament and the ends passed through the suture passages on the medial/ulnar aspect on the dorsal surface. The ends of these sutures are grasped and the implant is pulled down into position and the sutures are tightened.

In some embodiments, the implant can have a pair of suture passages on the medial and lateral sides of the implant to attach the dorsal and palmar ligaments respectively. In some embodiments, the suture passages can be on the dorsal surface of the implant. On the lateral side, the ends of the sutures can be passed through the suture passages from palmar to dorsal and tied on the dorsal side. On the medial/ulnar side, the suture passage can be dorsal only. The lateral suture grasps the distal aspect of the radioscapho ligament and each tail if passed though separated suture passages from palmar to dorsal. The implant is pulled down and the suture is tied dorsal. Then, the dorsal intercarpal ligament and the radio triquetrum ligament which were detached from the dorsal aspect of the triquetrum are suture into the suture passage on the medial/ulnar side of the implant. Both sutures can be tied adjacent the implant to stabilize and position the implant. In other embodiments, no sutures are used.

The disclosed implant can be fabricated from any biocompatible or biologically acceptable materials such as metals, synthetic polymers, ceramics and bone material and/or their composites and combinations thereof.

In some embodiments the metals can include stainless steel alloys, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan). The polymers can include polyethylene material, ultra-high molecular weight polyethylene or combinations thereof. Exemplary ceramics and composites thereof can include calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, or bone material. In one embodiment, the implant is ceramic. In other embodiments, the disclosed implant is a pyrocarbon. In some embodiments, the disclosed implant can be fabricated from graphite, pyrolytic carbon (e.g. PyroCarbon). In some embodiments, the disclosed implant is coated with desired biocompatible or biologically acceptable materials. In some embodiments, the implant is coated with ceramic or pyrolytic carbon. It should be understood to one of skill in the art that any material that has a modulus of elasticity similar to cortical bone can be used.

1. An wrist implant for replacement of the proximal carpal row, the implant comprising:
a single, one-piece integral body, the body having a substantially saddle-shaped or concave arcuate configuration comprising:
a dorsal surface;
a palmar surface;
a proximal end having a substantially convex shape;
a distal end configured to have three articulation surfaces and substantially opposing the proximal end, the distal end comprising;
a first lateral articulating surface defined by a first convex portion;
a second lateral articulating surface defined by second convex portion wherein the first lateral articulating surface is substantially opposing the second lateral articulating surface; and
a central articulating surface defined by a concave portion.

2. An wrist implant configured to replicate a proximal carpal row of a human wrist, the implant comprising:
a single, one-piece integral body, the body having a substantially saddle-shaped or concave arcuate configuration comprising:
a dorsal surface;
a palmar surface;
a proximal end having a substantially convex shape;
a distal end configured to have three articulation surfaces and substantially opposing the proximal end, the distal end comprising;
a first lateral articulating surface defined by a first convex portion;
a second lateral articulating surface defined by second convex portion wherein the first lateral articulating surface is substantially opposing the second lateral articulating surface; and
a central articulating surface defined by a concave portion.

3. A method for replacing a proximal carpal row of a human hand comprising
implanting a wrist implant, the wrist implant comprising:
a single, one-piece integral body, the body having a substantially saddle-shaped or concave arcuate configuration comprising:
a dorsal surface;
a palmar surface;
a proximal end having a substantially convex shape;
a distal end configured to have three articulation surfaces and substantially opposing the proximal end, the distal end comprising;
a first lateral articulating surface defined by a first convex portion;
a second lateral articulating surface defined by second convex portion wherein the first lateral articulating surface is substantially opposing the second lateral articulating surface; and
a central articulating surface defined by a concave portion.

4. A kit for replacing a proximal carpal row of a human hand comprising the proximal carpal row of claim 1.

5. The implant of claim 1, further comprising suture passages.

6. The implant of claim 1, wherein the suture passages are on the palmar-radial and dorsal-ulnar sides of the implant for ligament reattachment.

7. The implant of claim 1, wherein the dorsal and palmar surface are substantially planar.

8. The implant of claim 1, wherein the proximal surface is configured to articulate with a radius.

9. The implant of claim 1, wherein the first lateral articulating surface is configured to articulate with a trapezium and/or trapezoid.

10. The implant of claim 1, wherein the second lateral articulating surface is configured to articulate with a hamate.

11. The implant of claim 1, wherein the central articulating surface is configured to articulate with a capitate.

12. The implant of claim 1, wherein the implant is fabricated from ceramic.

13. The implant of claim 1, wherein the implant is fabricated from pyrocarbon.
